(19) United States
(12) Patent Application Publication
(10) Pub. No.: US 2026/0158120 A1
(43) Pub. Date: Jun. 11, 2026

(54) FUNGAL-DERIVED ENZYME COMPOSITION AND METHOD FOR TREATING EXOCRINE PANCREATIC INSUFFICIENCY

(71) Applicant: Henry Hilley, Denver, CO (US)

(72) Inventor: Henry Hilley, Denver, CO (US)

(21) Appl. No.: 19/411,641

(22) Filed: Dec. 8, 2025

Related U.S. Application Data
(60) Provisional application No. 63/730,327, filed on Dec. 10, 2024.

(51) Int. Cl. A61K 38/46; A61K 36/06; A61K 38/47; A61K 38/48

(52) U.S. Cl. CPC A61K 38/465; A61K 36/06; A61K 38/47; A61K 38/48

(57) ABSTRACT

A pharmaceutical composition and method for treating exocrine pancreatic insufficiency (EPI) comprising fungal fermentation-derived enzymes functionally analogous to endogenous digestive enzymes, including gastric lipase, amylase, and protease, stabilized with rice-derived silica. The composition delivers approximately 40,000 FIP lipase units per capsule without enteric coating, enabling immediate enzyme release and continuous activity across gastric (pH 1.5-3.5) and intestinal (pH 6-7.5) environments. The formulation maintains lipase-centered enzymatic proportionality to avoid protease over-exposure, provides flexible dosing of 1-5 capsules per meal, and supports empiric dose adjustment protocols based on symptom response. The composition treats EPI from multiple etiologies while supporting proper nutrient absorption through dual-phase digestive activity.

### CROSS REFERENCE TO RELATED APPLICATIONS

This application claims the benefit of U.S. Provisional Patent Application No. 63/730,327, filed Dec. 10, 2024, entitled “FUNGAL-DERIVED ENZYME COMPOSITION AND METHOD FOR TREATING EXOCRINE PANCREATIC INSUFFICIENCY,” the entire disclosure of which is incorporated herein by reference.

### FIELD OF THE INVENTION

The present invention relates generally to pharmaceutical compositions for treating digestive enzyme deficiencies, and more specifically to fungal fermentation-derived enzyme formulations for treating exocrine pancreatic insufficiency. The invention particularly addresses pancreatic enzyme replacement therapy that maintains enzymatic activity across both gastric and intestinal pH environments (pH 1.5-7.5) without requiring enteric coating protection, wherein the fungal-derived lipase is functionally analogous to endogenous human gastric lipase in its acid-stability profile, while providing optimal therapeutic ratios of lipase, protease, and amylase enzymes across multiple dosage strengths.

The invention further relates to methods of treating pancreatic insufficiency through empirical dose adjustment protocols that maintain consistent enzyme proportionality from prophylactic maintenance doses through high-concentration therapeutic formulations, thereby avoiding the excessive protease dosing problems inherent in existing enzyme replacement products.

### BACKGROUND OF THE INVENTION

Exocrine pancreatic insufficiency (EPI) represents a significant yet frequently underdiagnosed condition in gastroenterology. The condition occurs when the pancreas fails to produce and/or secrete adequate amounts of digestive enzymes, particularly lipase, amylase, and protease, which are essential for proper food digestion. This deficiency leads to inadequate breakdown of fats, carbohydrates, and proteins in the digestive tract.

The challenges in diagnosing and treating EPI are numerous. Current diagnostic methods, including the three-day stool test examining undigested fats, proteins, and fecal elastase test, demonstrate low sensitivity while maintaining high specificity. This diagnostic limitation means that while positive test results reliably confirm severe cases, mild to moderate cases frequently go undetected. The imprecise nature of available tests often leads to missed diagnoses, particularly in cases of mild to moderate insufficiency.

Conventional treatments for EPI typically involve prescription enzyme replacement therapy. However, these traditional treatments face several limitations. Current prescription enzyme products require special enteric coatings to protect the enzymes from stomach acid. These coatings, often containing phthalates, present challenges in achieving consistent dissolution in the small intestine, leading to unpredictable enzyme availability and potentially inconsistent therapeutic outcomes. The coating requirement results in variable dosing efficacy, as the protective layer may not dissolve uniformly or at the optimal location in the digestive tract. All FDA-approved pancreatic enzyme replacement therapies (PERTs) are enteric-coated porcine formulations, which has reinforced the industry assumption that high-dose therapeutic enzymes require protective coating to survive gastric transit. This paradigm has taught away from exploring acid-stable, coating-free enzyme systems for clinical EPI treatment.

The impact of untreated or inadequately treated EPI can be severe. Patients may experience a range of symptoms including abdominal pain, fecal urgency, and small intestinal bacterial overgrowth. The condition can lead to significant nutritional deficiencies as undigested food cannot be properly absorbed by the body. In severe cases, these nutritional deficiencies may contribute to serious health complications, including potential cognitive impacts due to inadequate fat absorption affecting brain nutrition.

Furthermore, the current market for digestive enzymes includes numerous over-the-counter supplements, but these products suffer from critical formulation deficiencies. Most OTC fungal enzyme supplements deliver fewer than 5,000 USP lipase units per capsule—an order of magnitude below the ˜40,000 USP lipase units per meal now recognized in clinical literature as the minimum therapeutic dose for EPI. A significant problem exemplified by existing vegan enzyme products such as Vital Nutrients Vegan Pancreatic Enzymes (containing approximately 12,500 FIP lipase and 100,000 HUT protease per capsule) is the disproportionate ratio of protease to lipase, creating an 8:1 protease-to-lipase ratio. This formulation defect creates a fundamental therapeutic problem: achieving adequate therapeutic lipase levels requires excessive protease dosing, which can cause severe stomach discomfort, nausea, and hospitalization-level sickness. Additionally, these products often contain undisclosed “enzyme blend” ingredients that may include wheat, soy, and other potential allergens, along with shelf stability issues that compromise therapeutic efficacy. The available supplements typically contain multiple additional ingredients and herbal extracts that may not contribute to addressing the core problem of enzyme insufficiency while creating potential confounding variables and adverse reactions.

By contrast, over-the-counter porcine enzyme supplements are typically uncoated and undergo rapid acid denaturation in gastric conditions, rendering them largely ineffective for therapeutic use. This dichotomy—enteric-coated prescription PERTs on one end and uncoated, inactive OTC porcine supplements on the other—has left no viable, non-prescription option for clinically meaningful enzyme therapy that addresses mild-to-moderate EPI or supports empiric treatment based on symptom presentation.

A critical unresolved problem in pancreatic enzyme replacement therapy is the maintenance of proper enzymatic proportionality across different dosage strengths. Existing products fail to maintain consistent lipase-to-protease ratios when patients require higher therapeutic doses, leading to a cyclical worsening of symptoms. Excessive protease doses cause severe gastrointestinal discomfort and can disrupt the microbiome balance in the small intestine, where undigested proteins from excessive protease activity can lead to overproduction of pathogenic bacteria, exacerbating the underlying pancreatic insufficiency condition 4. This proportionality problem represents a fundamental gap in current therapeutic approaches that has not been adequately addressed by existing enzyme replacement products.

The field requires improved solutions for treating EPI that can overcome these various limitations in current therapeutic approaches while providing more reliable and effective treatment options for patients suffering from this condition.

### SUMMARY OF THE INVENTION

An embodiment of the invention provides compositions and methods for treating exocrine pancreatic insufficiency (EPI) through a high-dose enzyme formulation derived from fungal fermentation. The preferred embodiment comprises a gastric lipase enzyme that maintains functionality in both stomach and small intestine environments without requiring protective enteric coating, along with complementary amylase and protease enzymes.

In an example of the invention, the composition delivers approximately 40,000 FIP lipase units per capsule as a base dose (where FIP and USP lipase units are functionally equivalent within ±10% for therapeutic comparison), with flexible dosing ranges from 40,000 to 200,000 units per meal achieved through the administration of one to five capsules. The preferred embodiment enables precise dose titration based on individual patient response and meal characteristics.

An embodiment of the invention utilizes a controlled fungal fermentation process to produce enzymes that are functionally identical to endogenous human digestive enzymes. The preferred embodiment maintains a simple formulation approach, incorporating only the essential enzymatic components and a rice-derived silica stabilizer within a standard pharmaceutical-grade capsule.

The preferred embodiment demonstrates dual-environment functionality, maintaining enzymatic activity across the pH range found in the stomach (pH 1.5-3.5) and small intestine (pH 6-7.5). An example of the invention provides shelf stability for 24 months under standard storage conditions while maintaining enzyme activity levels.

An embodiment of the invention addresses various manifestations of EPI across a spectrum of severity levels, from prophylactic support to high-dose therapeutic intervention, while maintaining consistent enzymatic proportionality. The invention provides three distinct formulation approaches: a prophylactic formulation for maintenance therapy containing approximately 20,000 FIP lipase units with proportionally balanced protease and amylase; the base therapeutic formulation providing 40,000 USP lipase units; and a high-concentration liquid suspension formulation delivering 160,000-200,000 FIP lipase units for patients requiring maximum therapeutic support. Critically, across all dosage levels of the formulation, the proportionality remains consistent. The formulation is optimized for lipase-centered proportionality, delivering therapeutically high lipase activity relative to supporting protease and amylase levels to ensure balanced digestion without protease over-exposure. In specific embodiments, the relative activities approximate a 1:16:4.5 lipase:protease:amylase ratio (USP basis). This proportional consistency prevents the severe gastrointestinal discomfort associated with unnecessarily high protease levels and supports proper microbiome balance by providing pre-digestion of proteins within the stomach, ensuring that food reaches the small intestine in an appropriately processed state.

### DETAILED DESCRIPTION OF THE INVENTION

An embodiment of the invention provides compositions and methods for treating exocrine pancreatic insufficiency (EPI) through enzyme replacement therapy. The preferred embodiment comprises a high-dose enzyme formulation derived through fungal fermentation processes, which produces enzymes functionally analogous to those naturally occurring in the human digestive system. In particular, an example of the invention includes a gastric lipase enzyme that maintains functionality in both the stomach and small intestine environments without requiring protective enteric coating.

The preferred embodiment of the invention delivers therapeutic doses of digestive enzymes in a simple capsule format, enabling flexible dosing protocols based on individual patient needs and symptom presentation. An embodiment of the invention provides enzyme concentrations significantly higher than traditional over-the-counter supplements, specifically formulated to address clinical enzyme insufficiency. The composition maintains stability through the inclusion of minimal excipients, focusing on the active enzymatic components while avoiding unnecessary additives or herbal supplements commonly found in conventional digestive aids.

In an example of the invention, the formulation enables direct enzyme activity in the stomach, providing immediate digestive support upon ingestion, while maintaining functionality as food passes into the small intestine. This dual-location activity represents a departure from traditional enzyme replacement approaches that limit enzyme availability to the small intestine environment. The preferred embodiment allows for empiric dose adjustment protocols, enabling healthcare providers to optimize treatment based on individual patient response and symptom resolution.

In a preferred embodiment of the invention, the enzyme composition comprises a gastric lipase derived through fungal fermentation processes. The fungal fermentation produces a lipase enzyme that is functionally identical to human gastric lipase, enabling activity in both stomach and small intestine environments. The fermentation process utilizes selected fungal strains cultured under controlled conditions to produce the desired enzyme profile.

An example of the invention includes amylase enzyme produced through similar fermentation processes, with activity levels optimized for carbohydrate digestion throughout the digestive tract. The protease enzyme component in an embodiment of the invention is likewise produced via fermentation methods, providing protein-digesting capability that remains stable in varying pH environments.

The preferred embodiment utilizes rice-derived silica as a stabilizing excipient. The silica component serves to maintain enzyme stability during storage and protect against moisture-induced degradation. The stabilizer comprises approximately 1-5% by weight of the total composition.

In an embodiment of the invention, the enzymes and stabilizer are contained within a standard pharmaceutical-grade capsule composed of hydroxypropyl methylcellulose (HPMC) or similar acceptable materials. The capsule size is selected to accommodate the therapeutic dose while maintaining ease of administration. The capsule serves as the primary delivery vehicle without additional coatings or barriers.

The preferred embodiment specifically excludes enteric coating, allowing for immediate enzyme release upon capsule dissolution. This enables rapid onset of enzymatic activity in the stomach environment while maintaining functionality as the composition transitions to the small intestine. The absence of enteric coating represents a departure from traditional enzyme delivery systems that rely on pH-dependent release mechanisms.

An example of the invention provides the following approximate composition ranges per capsule:
Lipase: 35,000-45,000 USP units, and in one exemplary embodiment 40,000 USP
Amylase: 15,000-35,000 USP units, and in one exemplary embodiment 18,026.4 USP
Protease: 600,000-700,000 USP units, and in one exemplary embodiment 650,000 USP
Rice-derived silica: 1-5% w/w
Capsule shell: Standard pharmaceutical grade HPMC

An example of the invention provides specific enzyme composition ranges per capsule that represent significant advances over prior art approaches. The lipase content of 35,000-45,000 USP units, with an exemplary embodiment of 40,000 USP units, enables therapeutic enzyme delivery that more closely approximates physiological needs compared to traditional supplements.

The amylase content ranges from 15,000-35,000 USP units, with an exemplary embodiment of 18,026.4 USP units, providing complementary carbohydrate digestion support throughout the digestive tract.

The protease content of 600,000-700,000 USP units (100,000 HUT), with an exemplary embodiment of 650,000 USP units, represents a substantial increase in proteolytic enzyme activity compared to conventional enzyme replacement products.

This enhanced protease activity, combined with the formulation's ability to maintain enzymatic function across both gastric and intestinal environments (pH 1.5-7.5), enables more comprehensive protein digestion throughout the digestive tract. The significantly higher protease content supports optimal protein breakdown and absorption while maintaining the formulation's simplified approach that excludes unnecessary additives or herbal supplements.

The invention in an embodiment provides a comprehensive multi-tier formulation approach that addresses the full spectrum of pancreatic insufficiency severity while maintaining consistent enzymatic proportionality across all dosage levels. This approach solves the critical problem identified in existing enzyme products where achieving therapeutic lipase levels requires excessive and harmful protease dosing.

Prophylactic Formulation: An embodiment of the invention provides a maintenance-level formulation containing approximately 20,000 FIP units of lipase, 50,000 HUT units of protease, and 1,877.75 SKB units of amylase per half-sized capsule. This prophylactic formulation maintains the optimal lipase-to-protease ratio while providing enzyme support for individuals with mild pancreatic insufficiency or those requiring maintenance therapy. The formulation includes rice-derived silica (Nu-Flow) stabilizer for enhanced shelf stability, addressing the stability problems found in existing vegan enzyme products.

High-Dose Liquid Suspension: For patients requiring maximum therapeutic intervention (typically 4-5 capsules per meal of the base formulation), the invention provides a liquid suspension formulation containing 160,000-200,000 FIP units of lipase and 300,000-400,000 HUT units of protease per dose. This liquid formulation addresses the psychological pill burden problem while maintaining the same therapeutic proportionality as the capsule formulations. The liquid formulation may be encapsulated in gel capsules to prevent mucosal irritation, as the enzyme powder cannot be safely removed from capsules due to its potency.

Proportionality Maintenance: A critical aspect of the invention is that across all dosage levels—prophylactic, therapeutic, and high-dose—the proportionality of enzymes remains consistent. This consistency ensures that patients can achieve adequate lipase levels without the excessive protease intake that characterizes existing products such as Vital Nutrients Vegan Pancreatic Enzymes. The maintained proportionality provides pre-digestion of proteins within the stomach, reducing the burden on the compromised pancreas and preventing the microbiome imbalance that can cyclically worsen small intestinal problems associated with pancreatic insufficiency.

In a preferred embodiment of the invention, each capsule contains 40,000 USP units of lipase as the base therapeutic dose. This standardized unit measurement follows established pharmacological conventions for expressing enzyme activity levels.

An example of the invention provides flexible dosing ranges from 40,000 to 200,000 USP units per meal, achieved through variable capsule administration. The preferred embodiment enables precise dose titration through the administration of 1-5 capsules per meal, allowing for individualized treatment protocols based on symptom presentation and severity.

In an embodiment of the invention, the dosing protocol follows a stepwise approach:
Initial dose: One capsule (40,000 USP units) per meal
Intermediate dose: Two to three capsules (80,000-120,000 USP units) per meal
Maximum dose: Five capsules (320,000 USP units) per meal

The preferred embodiment allows for dose adjustment based on meal size and fat content, with larger or higher-fat meals potentially requiring increased enzyme supplementation. An example of the invention includes specific dosing recommendations:
Small meals (<500 calories): 1-2 capsules
Medium meals (500-800 calories): 2-3 capsules
Large meals (>800 calories): 3-5 capsules

An embodiment of the invention provides for dose timing flexibility, with capsules administered immediately before or during meal consumption to optimize enzyme availability during digestion.

In a preferred embodiment of the invention, the enzyme composition demonstrates dual-environment functionality, maintaining active enzymatic properties in both the acidic conditions of the stomach (pH 1.5-3.5) and the more neutral environment of the small intestine (pH 6-7.5). An example of the invention provides consistent enzyme activity across this pH range, enabling continuous digestive support throughout the gastrointestinal tract.

The preferred embodiment exhibits particular stability in acidic stomach conditions, with the fungal-derived gastric lipase maintaining structural integrity and catalytic function at low pH levels. An embodiment of the invention demonstrates resistance to acid-induced denaturation, allowing for immediate enzymatic activity upon release from the capsule delivery system.

In an example of the invention, the inherent stability of the fungal-derived enzymes eliminates the need for protective enteric coating. The preferred embodiment achieves therapeutic efficacy through direct release of active enzymes, contrasting with traditional formulations that require pH-dependent coating systems for enzyme protection. The industry-wide reliance on enteric-coated porcine formulations for all FDA-approved PERTs has created a strong teaching-away from coating-free, high-dose enzyme systems, as the prevailing belief held that therapeutic potencies require protection from gastric acid.

An embodiment of the invention maintains stability under standard storage conditions (15-25° C., relative humidity≤60%) for a minimum of 24 months. The preferred embodiment demonstrates the following stability characteristics:
Enzyme activity retention: ≥90% after 24 months
Moisture sensitivity: Stable in sealed capsules at ≤60% relative humidity
Temperature stability: Maintains activity between 15-25° C.
pH stability: Retains function across pH range 1.5-7.5

The shelf-stable formulation in an example of the invention is achieved through the combination of inherently stable fungal-derived enzymes and the incorporation of rice-derived silica as a stabilizing excipient.

In a preferred embodiment of the invention, the enzyme composition is produced through a controlled fungal fermentation process utilizing selected fungal strains. An example of the invention employs batch fermentation under the following general conditions:
Temperature range: 25-35° C.
pH range: 4.5-6.5
Dissolved oxygen: 20-40% saturation
Fermentation duration: 5-7 days

The preferred embodiment produces gastric lipase through fermentation that is functionally identical to endogenous human gastric lipase, demonstrating comparable enzymatic activity profiles and pH stability characteristics.

In a preferred embodiment of the invention, specific strain selection for fungal fermentation is achieved through identification and cultivation of fungal strains that produce lipase enzymes demonstrating structural and functional characteristics matching endogenous human gastric lipase.

The selected strains are evaluated for their ability to produce enzymes that maintain catalytic activity across the physiological pH range of 1.5-7.5 and demonstrate stability in both gastric and intestinal environments. The strain selection process prioritizes organisms capable of high-yield enzyme production while maintaining consistent quality and purity in controlled fermentation conditions of 25-35° C., pH 4.5-6.5, and 20-40% dissolved oxygen saturation. The selected strains must reliably generate lipase that exhibits therapeutic efficacy without requiring protective enteric coating, distinguishing it from traditional porcine-derived enzymes that are susceptible to acid denaturation. Quality control testing verifies that the selected strains consistently produce enzymes meeting specified activity levels and stability parameters.

In a preferred embodiment of the invention, optimized growth conditions are maintained through precise control of fermentation parameters including temperature range of 25-35° C., pH range of 4.5-6.5, dissolved oxygen at 20-40% saturation, and fermentation duration of 5-7 days.

These conditions are continuously monitored and adjusted to ensure optimal enzyme production and activity levels throughout the fermentation process.

The controlled fermentation parameters include standardized protocols for media composition, inoculation procedures, and growth phase monitoring to achieve consistent enzyme yields and quality across production batches.

The fermentation process is validated through in-process testing of enzyme activity, purity, and stability to ensure the resulting lipase maintains functionality across the target pH range of 1.5-7.5.

Standardized harvesting protocols involve separation of the enzyme from the fermentation broth through controlled filtration and/or centrifugation steps, followed by purification and concentration to achieve desired enzyme activity levels.

The harvested enzyme complex undergoes quality control testing to verify enzyme activity levels, purity specifications, stability parameters, and microbial limits before final formulation. This standardized approach ensures batch-to-batch consistency in enzyme potency and purity while maintaining the functional characteristics matching endogenous human gastric lipase.

In an example of the invention, post-fermentation processing begins with separation of the enzyme complex from the fermentation broth using controlled separation techniques that maintain enzyme stability and activity.

The separated enzyme material undergoes purification through a combination of filtration and/or centrifugation steps to remove cellular debris and other fermentation byproducts while preserving the functional characteristics of the lipase enzyme. Following purification, the enzyme preparation is concentrated to achieve the specified activity levels required for therapeutic dosing, targeting 35,000-45,000 USP units of lipase per capsule in the final formulation.

The concentration process is carefully controlled to maintain enzyme stability and prevent degradation of activity. The concentrated enzyme preparation undergoes standardization to ensure consistent and precise enzyme unit measurements following established pharmacological conventions for expressing enzyme activity levels.

Quality control testing verifies the standardized enzyme activity meets specifications before proceeding to final formulation steps. This standardized approach enables reliable batch-to-batch consistency in enzyme potency while maintaining the functional characteristics matching endogenous human gastric lipase.

The preferred embodiment maintains a simple formulation approach that specifically excludes additional herbs, botanical extracts, and non-essential additives to focus on the core therapeutic components.

The final formulation in an exemplary embodiment contains only:

The purified enzyme complex, comprising the fungal-derived gastric lipase, amylase and protease enzymes that have been separated, purified and standardized through controlled post-fermentation processing to achieve specified activity levels. The enzyme complex maintains stability and functionality across both gastric and intestinal pH ranges without requiring protective enteric coating.

Rice-derived silica stabilizer comprising approximately 1-5% by weight of the total composition, which serves to maintain enzyme stability during storage and protect against moisture-induced degradation. The stabilizer is specifically selected to support long-term product stability while avoiding unnecessary excipients.

A standard pharmaceutical-grade capsule shell composed of hydroxypropyl methylcellulose (HPMC) or similar acceptable materials, selected to accommodate the therapeutic dose while maintaining ease of administration. The capsule serves as the primary delivery vehicle in an embodiment without additional coatings or barriers, enabling immediate enzyme release upon dissolution.

This simplified formulation approach represents a departure from traditional enzyme replacement products that often contain multiple additional ingredients and herbal extracts that may not contribute to addressing the core problem of enzyme insufficiency. The exclusion of non-essential components helps maintain product stability while focusing on the key therapeutic elements needed for treating EPI.

The invention in an embodiment maintains formulation purity by specifically excluding problematic additives found in existing enzyme products. Unlike competing products such as Vital Nutrients Vegan Pancreatic Enzymes, which contain enzyme blend ingredients that may include wheat, soy, and other potential allergens, the present formulation contains only the essential enzymatic components and the rice-derived silica stabilizer. This purity approach eliminates potential allergen exposure and reduces the risk of adverse reactions from unnecessary additives.

The rice-derived silica stabilizer (Nu-Flow) provides superior shelf stability compared to existing vegan enzyme products, which are known to suffer from stability issues that compromise therapeutic efficacy. The stabilizer maintains enzyme activity under standard storage conditions while avoiding the need for chemical coatings or preservatives that may interfere with enzyme function or cause adverse reactions.

The manufacturing process in an example of the invention concludes with comprehensive quality control testing to verify multiple critical parameters.

Enzyme activity levels are evaluated to confirm the specified therapeutic potency is achieved, particularly verifying the lipase content of 35,000-45,000 USP units per capsule and corresponding amylase and protease activities.

Purity specifications are assessed through standardized testing protocols to ensure the final formulation contains only the intended enzyme components, specifically confirming the absence of fermentation byproducts or other contaminants.

The testing verifies the composition maintains the simplified formulation approach without additional non-essential additives. Stability parameters are evaluated to confirm the formulation maintains enzyme activity under standard storage conditions of 15-25° C. and relative humidity≤60% for a minimum of 24 months. Testing verifies retention of >90% enzyme activity after 24 months and stability across the physiological pH range of 1.5-7.5.

Microbial limits testing ensures the final product meets pharmaceutical quality standards for microbial content. This testing is particularly important given the fermentation-based manufacturing process, verifying the purification steps have effectively removed any potential microbial contamination while preserving the desired enzyme activity.

In a preferred embodiment of the invention, the enzyme composition is administered with meals to optimize digestive support. An example of the invention provides for dosing immediately before or during food consumption to ensure enzyme availability during the digestive process.

In a preferred embodiment of the invention, treatment follows an empiric dosing protocol with three distinct phases to optimize therapeutic outcomes.

The Initial Phase spanning weeks 1-2 begins with a starting dose of one capsule (40,000 USP units) per meal to establish baseline response. During this period, patients undergo careful monitoring to document initial therapeutic response and any changes in symptoms including steatorrhea, GI discomfort, and nutritional parameters. Documentation includes tracking of stool characteristics, abdominal pain, bloating, and other digestive symptoms.

The Adjustment Phase occurs during weeks 2-6, allowing for dose optimization based on individual patient response. The dose may be incrementally increased by one capsule per meal as needed to achieve symptom control, not exceeding five capsules (200,000 USP units) per meal. Weekly assessments evaluate symptom improvement, focusing on resolution of malabsorption indicators and quality of life measures. This structured dose titration enables precise adjustment to meet individual therapeutic requirements.

The Maintenance Phase establishes optimal long-term dosing once symptom control is achieved.

Regular monitoring continues to assess ongoing symptom control, nutritional status, and treatment efficacy. Periodic reassessment of dose requirements allows for adjustments based on changes in patient condition or meal characteristics. The maintenance protocol includes evaluation of fat-soluble vitamin levels, weight maintenance, and other markers of nutritional status to ensure sustained therapeutic benefit.

The invention's dramatically increased dosing in accordance with an embodiment represents a significant departure from prior art approaches in several key ways. While traditional pancreatic enzyme replacement therapy typically delivers 25% or less of the recommended dose, the present invention enables delivery of 40,000-200,000 USP units of lipase per meal through flexible dosing of 1-5 capsules. This higher dosing range more closely approximates physiological needs, as a normal pancreas produces approximately 900,000 USP units of lipase during an average meal, with approximately 90,000 units needed to prevent steatorrhea. The invention's base dose of 40,000 USP units per capsule in the preferred embodiment, which can be increased to 200,000 units through multiple capsule administration, provides significantly greater enzyme delivery compared to conventional over-the-counter supplements. This approach differs from prior art in several key ways. While traditional PERT typically relies on rigid weight-based dosing protocols (500 to 2,500 lipase units per kg per meal), the present invention enables precise, empiric dose titration from 40,000 to 200,000 USP units based on individual symptom resolution and clinical response, rather than solely on body weight calculations. This enhanced dosing capability, combined with the formulation's ability to maintain activity in both gastric and intestinal environments without requiring protective enteric coating, enables more effective treatment of clinical enzyme insufficiency compared to traditional approaches that limit enzyme availability to the small intestine environment.

An example of the invention includes comprehensive monitoring through patient symptom diaries that track key indicators including steatorrhea, abdominal pain, digestive discomfort, and changes in stool characteristics. Weekly assessment of digestive function evaluates resolution of malabsorption symptoms, including monitoring of stool frequency, consistency, and visible fat content, along with tracking of weight, muscle mass and strength. Documentation of adverse effects includes monitoring for potential gastrointestinal symptoms such as nausea, bloating, flatulence and abdominal cramping, while also tracking any allergic reactions or other complications. Evaluation of nutritional status encompasses regular assessment of anthropometric measures, serum nutritional parameters including fat-soluble vitamins A, D, E and K, proteins such as prealbumin and albumin, and essential micronutrients. This continuous monitoring protocol enables optimization of enzyme replacement therapy through evidence-based dose adjustments while ensuring early detection of any nutritional deficiencies or adverse effects that may require intervention.

The preferred embodiment enables individualized dose adjustment based on specific clinical responses and biomarkers. Resolution of acute abdominal pain associated with eating is monitored through patient reporting, while chronic symptoms including systemic inflammation, generalized discomfort, diarrhea, and fecal incontinence are tracked through ongoing assessment. Dose adjustments are made to optimize both acute and chronic symptom relief while maintaining digestive function. Improvement in stool consistency is evaluated through monitoring changes in steatorrhea, formed versus loose stools, and visible fat content, allowing for precise dose titration based on these objective markers.

Reduction in digestive discomfort is assessed through tracking of symptoms including bloating, flatulence, and postprandial fullness, with dose modifications made to achieve optimal symptom control. Enhanced nutrient absorption is verified through monitoring of serum nutritional parameters including fat-soluble vitamins, proteins such as prealbumin and albumin, and essential micronutrients, enabling evidence-based dose optimization.

This approach differs from prior art in several key ways. While traditional pancreatic enzyme replacement therapy typically relies on weight-based dosing protocols (500 to 2,500 lipase units per kg per meal), the present invention in an embodiment enables precise dose titration from 40,000 to 200,000 USP units based on individual symptom resolution and clinical response. Additionally, unlike conventional treatments that focus primarily on steatorrhea reduction and weight-based calculations, this invention's comprehensive symptom monitoring and biomarker assessment in an embodiment allows for more targeted therapy optimization based on actual patient outcomes. The ability to adjust dosing based on both clinical symptoms and nutritional parameters, rather than solely body weight calculations, represents a significant advance over existing approaches that lack such systematic response-driven protocols. Furthermore, the invention's dual-location activity in both stomach and small intestine environments in an embodiment enables more complete symptom resolution compared to traditional enteric-coated formulations that only activate in the small intestine.

An embodiment of the invention provides for long-term monitoring to ensure sustained therapeutic benefit and optimal dose maintenance. In an embodiment of the invention, the enzyme composition is specifically formulated for the treatment of Exocrine Pancreatic Insufficiency (EPI), a condition characterized by inadequate pancreatic enzyme production and secretion. An example of the invention addresses both diagnosed EPI cases and suspected cases where empiric therapy may be warranted. The invention addresses EPI arising from multiple etiologies, including but not limited to: chronic pancreatitis, post-surgical pancreatic insufficiency, cystic fibrosis, pancreatic cancer, diabetes-associated exocrine insufficiency, age-related pancreatic hypofunction, celiac disease with secondary insufficiency, inflammatory bowel disease, and genetic conditions affecting pancreatic function including Shwachman-Diamond syndrome and Johanson-Blizzard syndrome.

In an embodiment of the invention, the enzyme composition demonstrates significant efficacy in addressing multiple aspects of pancreatic insufficiency. Steatorrhea symptoms are effectively managed through the dual-environment functionality of the fungal-derived enzymes, which maintain enzymatic activity in both stomach and small intestine environments to optimize fat digestion and absorption. The formulation resolves irregular bowel movements through enhanced digestive enzyme activity throughout the gastrointestinal tract, improving both the frequency and consistency of bowel movements compared to baseline.

Fecal urgency and other manifestations of EPI are addressed through improved nutrient digestion and absorption, reducing the osmotic load in the intestine that can trigger urgent bowel movements.

The invention in an embodiment has been observed by the inventor to prevent malnutrition, unexplained weight loss, and sarcopenia by enabling proper digestion and absorption of nutrients that would otherwise be unavailable to the body. For patients with gastroparesis, the formulation's ability to begin digestion immediately in the stomach helps break down food and promote gastric emptying, facilitating passage of nutrients into the small intestine.

The invention in an embodiment has been observed by the inventor to help reduce risk factors associated with cognitive decline, as proper fat digestion and absorption is essential for brain health, with the brain requiring 20-30% of dietary fat and fat-soluble vitamins for optimal function.

This is particularly relevant given that pancreatic function naturally decreases after age 50, representing a significant population with mild EPI that may benefit from enzyme supplementation. The formulation's high lipase content (40,000 USP/FIP units) and dual-environment functionality support comprehensive fat digestion and absorption, helping ensure adequate nutrient delivery for both immediate metabolic needs and long-term cognitive health.

The invention in accordance with various embodiments effectively treats malabsorption indicators through multiple mechanisms. The high-dose enzyme delivery of 40,000-200,000 USP units of lipase enables proper fat absorption and utilization of fat-soluble vitamins. The formulation's ability to maintain activity across varying pH environments (1.5-7.5) ensures consistent enzyme availability throughout digestion, supporting proper nutrient absorption and reducing malabsorption symptoms. Regular monitoring of nutritional parameters, including fat-soluble vitamins, proteins, and essential micronutrients, confirms improvement in absorption markers and guides ongoing dose optimization.

This comprehensive approach to treating pancreatic insufficiency represents an advance over prior art, which typically focuses primarily on steatorrhea reduction through delayed enzyme release in the small intestine.

The invention's immediate enzyme availability and sustained activity throughout the digestive tract in an embodiment enables more complete resolution of the multiple manifestations of pancreatic insufficiency.

An embodiment of the invention provides relief from heartburn symptoms through enhanced gastric digestion, particularly of fats and proteins. The preferred embodiment achieves this through immediate enzymatic activity in the stomach environment, supporting normal digestive processes.

In an example of the invention, upper abdominal pain management is achieved through multiple complementary mechanisms.

Enhanced food breakdown in the stomach is enabled by the immediate release and activation of enzymes upon capsule dissolution, with the fungal-derived lipase maintaining full functionality in the acidic gastric environment. This early-stage digestion represents an advance over traditional enteric-coated formulations that remain inactive until reaching the small intestine.

The reduced digestive burden results from the high enzyme concentrations (40,000-200,000 USP units of lipase) supporting comprehensive nutrient breakdown throughout the digestive tract.

The formulation's ability to maintain enzymatic activity across both gastric and intestinal pH ranges (1.5-7.5) ensures consistent digestive support, reducing the workload on the compromised pancreas. Improved nutrient absorption is achieved through the dual-environment functionality that enables proper digestion and absorption of fats, proteins and carbohydrates.

The formulation's ability to begin digestion in the stomach and maintain activity through the small intestine supports optimal nutrient utilization while minimizing undigested food. Decreased gastric retention is supported by enhanced gastric digestion that promotes proper gastric emptying.

The immediate enzyme availability upon capsule dissolution helps normalize gastric motility by reducing the accumulation of undigested food in the stomach. This represents an improvement over conventional enzyme replacements that delay enzyme activation until the small intestine, potentially prolonging gastric retention of undigested nutrients.

An embodiment of the invention demonstrates particular efficacy in addressing the spectrum of EPI manifestations, from mild to moderate cases that may be missed by traditional diagnostic methods. The preferred embodiment demonstrates effectiveness in resolving multiple digestive issues related to enzyme deficiency.

Nutrient malabsorption is addressed through the formulation's ability to maintain enzymatic activity across both gastric and intestinal environments (pH 1.5-7.5), enabling proper digestion and absorption of fats, proteins, and essential nutrients throughout the digestive tract. The high-dose enzyme delivery of 40,000-200,000 USP units supports comprehensive nutrient absorption by providing sufficient enzymatic activity to break down dietary components.

Bacterial overgrowth is managed through improved digestive efficiency and reduced substrate availability for bacterial fermentation. The immediate enzyme release upon capsule dissolution, combined with activity in both stomach and small intestine environments, helps prevent the accumulation of undigested nutrients that can promote harmful bacterial proliferation. By optimizing digestion and absorption of nutrients throughout the digestive tract, the formulation reduces the availability of undigested food that would otherwise serve as substrate for pathogenic bacteria. This enhanced digestive efficiency helps restore and maintain a healthy balance of intestinal microbiota by allowing beneficial bacteria to thrive while limiting resources for harmful bacterial overgrowth. The dual-environment functionality and high enzyme concentrations (40,000-200,000 USP/FIP units) support comprehensive nutrient breakdown and absorption, creating conditions that favor beneficial bacterial populations through competitive inhibition of pathogenic organisms.

The immediate enzyme release upon capsule dissolution, combined with activity in both stomach and small intestine, helps prevent the accumulation of undigested nutrients that can promote bacterial proliferation.

Gastric motility challenges are addressed through enhanced digestion beginning in the stomach environment, supporting proper gastric emptying and reducing retention of undigested food.

The formulation's dual-environment functionality helps normalize the digestive process and gastric transit.

Post-meal discomfort is relieved through multiple mechanisms including improved nutrient breakdown, reduced gastric retention, and enhanced absorption. The formulation's immediate enzyme release and dual-environment functionality helps reduce pancreatic strain by supplementing natural enzyme production, potentially alleviating upper abdominal pain associated with inadequate pancreatic enzyme secretion. This upper abdominal pain, which is often misdiagnosed as gallbladder-related discomfort, can be chronic and particularly pronounced after meals when the compromised pancreas struggles to produce sufficient digestive enzymes. The high enzyme concentrations (40,000-200,000 USP/FIP units) provided by the invention support comprehensive nutrient digestion while reducing the secretory burden on the pancreas. Clinical evidence of symptom resolution through empiric therapy demonstrates the formulation's effectiveness in relieving post-meal discomfort through these multiple complementary mechanisms. The dual-location activity in both stomach and small intestine environments enables more complete digestive support compared to traditional enteric-coated formulations that only activate in the small intestine.

The formulation's ability to begin digestion immediately upon release, rather than waiting for small intestine activation as with traditional enteric-coated products, helps minimize postprandial symptoms. Regular monitoring of symptom response enables dose optimization to achieve maximal relief of post-meal discomfort while maintaining proper digestion and absorption.

This comprehensive approach represents an advance over prior art enzyme replacements that typically provide insufficient enzyme concentrations and delayed activation, limiting their ability to fully address the spectrum of digestive issues associated with enzyme deficiency.

The present invention in an embodiment specifically addresses and solves critical deficiencies in existing enzyme replacement products. Existing vegan enzyme products, exemplified by Vital Nutrients Vegan Pancreatic Enzymes, suffer from a fundamental formulation flaw: disproportionately high protease content relative to lipase content (100,000 HUT protease to only 12,500 FIP lipase). This imbalanced ratio creates a therapeutic impossibility—achieving adequate lipase levels for pancreatic insufficiency requires excessive protease dosing that causes severe gastrointestinal distress, nausea, and potential hospitalization.

The present invention solves this proportionality problem in an embodiment by maintaining optimal lipase-to-protease ratios across all formulation strengths. This consistent proportionality enables patients to achieve therapeutic lipase levels without the excessive protease intake that characterizes existing products. The balanced enzyme ratios support proper gastric pre-digestion of proteins, preventing the microbiome imbalance that occurs when excessive undigested proteins reach the small intestine, where they can promote pathogenic bacterial overgrowth and cyclically worsen pancreatic insufficiency symptoms.

Additionally, the present invention in an embodiment provides superior formulation purity and stability compared to existing products. Unlike products containing undisclosed “enzyme blends” with potential wheat, soy, and other allergens, the present formulation maintains transparency and purity with only essential enzymatic components and a rice-derived stabilizer. The Nu-Flow stabilizer provides shelf stability advantages over existing vegan enzyme products that suffer from degradation issues, ensuring consistent therapeutic potency throughout the product's shelf life.

Without wishing to be bound by theory, it has been unexpectedly observed that the present formulation may reduce functional strain on the pancreas by initiating enzymatic digestion in the stomach before chyme enters the duodenum. In contrast to conventional enteric-coated preparations that delay enzyme release until the intestinal phase, the fungal-derived gastric lipase of the present invention provides immediate lipid hydrolysis in the gastric environment. This early-phase activity appears to lower the required endogenous pancreatic enzyme secretion to achieve equivalent nutrient absorption.

Such predigestion within the stomach was not anticipated by prior art formulations, which uniformly assumed that acid exposure would inactivate therapeutic enzymes or that gastric-phase lipolysis was unnecessary for clinical efficacy. The ability of the composition to maintain high catalytic function under gastric conditions (pH 1.5-3.5) while continuing activity through intestinal transit therefore yields an unpredicted physiological advantage: continuous digestion from stomach through intestine, potentially improving fat assimilation and decreasing postprandial pancreatic workload.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

I claim:

1. A pharmaceutical composition for treating exocrine pancreatic insufficiency comprising:
a fungal fermentation-derived gastric lipase;
an amylase enzyme;
a protease enzyme; and
a rice-derived silica stabilizer;
wherein the composition is encapsulated without an enteric coating.

2. The pharmaceutical composition of claim 1, wherein the gastric lipase is present in an amount of 35,000-45,000 USP units per capsule.

3. The pharmaceutical composition of claim 1, wherein the composition maintains enzymatic activity across a pH range of 1.5-7.5.

4. The pharmaceutical composition of claim 1, wherein the composition maintains at least 90% enzyme activity after 24 months of storage at 15-25° C. and relative humidity≤60%.

5. The pharmaceutical composition of claim 1, wherein the composition comprises a prophylactic formulation containing approximately 20,000 FIP units of lipase, 50,000 HUT units of protease, and 1,877.75 SKB units of amylase per dosage unit.

6. The pharmaceutical composition of claim 1, wherein the composition comprises a high-dose formulation containing 160,000-200,000 FIP units of lipase and 300,000-400,000 HUT units of protease per dosage unit.

7. The pharmaceutical composition of claim 6, wherein the high-dose formulation is provided as a liquid suspension.

8. The pharmaceutical composition of claim 7, wherein the liquid suspension is encapsulated in a gel capsule.

9. The pharmaceutical composition of claim 1, wherein the composition specifically excludes wheat-derived and soy-derived ingredients.

10. The pharmaceutical composition of claim 1, wherein the composition consists essentially of the fungal fermentation-derived gastric lipase, amylase enzyme, protease enzyme, and rice-derived silica stabilizer, excluding enzyme blends with undisclosed ingredients.

11. The pharmaceutical composition of claim 1, wherein the composition is formulated as a liquid suspension, emulsion, or powder suitable for reconstitution, delivering equivalent enzymatic activity to the capsule formulation.

12. The pharmaceutical composition of claim 1, further comprising one or more digestive cofactors selected from the group consisting of bile salts, betaine HCl, and pepsin.

13. The pharmaceutical composition of claim 1, wherein the composition excludes herbal extracts and additional non-enzyme additives.

14. The pharmaceutical composition of claim 1, wherein the fungal fermentation-derived gastric lipase is functionally identical to endogenous human gastric lipase.

15. A dosing kit for treating exocrine pancreatic insufficiency comprising: two or more dosage strengths of the pharmaceutical composition of claim 1, and instructions for empiric dose titration based on symptom severity.

16. A method of treating exocrine pancreatic insufficiency comprising:
administering to a patient in need thereof a therapeutically effective amount of the pharmaceutical composition of claim 1 with each meal.

17. The method of claim 16, wherein the therapeutically effective amount comprises 40,000-200,000 USP units of lipase per meal.

18. The method of claim 16, further comprising:
initiating treatment with one capsule per meal;
monitoring patient symptoms; and
adjusting dosage based on symptom response.

19. The method of claim 16, wherein the dosing is titrated empirically based on patient-reported symptom resolution including reduction of steatorrhea, bloating, fecal urgency, and upper abdominal pain, and wherein the dosage is adjusted at intervals of one to two weeks during an initial titration phase.

20. A method of manufacturing a pharmaceutical composition comprising:
producing gastric lipase through fungal fermentation;
combining the gastric lipase with amylase enzyme, protease enzyme, and rice-derived silica; and
encapsulating the combination without an enteric coating.

21. The method of claim 20, wherein the fungal fermentation is conducted under conditions comprising:
temperature range of 25-35° C.;
pH range of 4.5-6.5;
dissolved oxygen of 20-40% saturation; and
fermentation duration of 5-7 days.
